(19) United States
(12) Patent Application Publication
(10) Pub. No.: US 2022/0040244 A1
(43) Pub. Date: Feb. 10, 2022

(54) PROBIOTICS FOR COGNITIVE AND MENTAL HEALTH

(71) Applicant: DUPONT NUTRITION BIOSCIENCES APS, Copenhagen (DK)

(72) Inventors: MARKUS LEHTINEN, Kantvik (FI); Yuejian Mao, CHANGNING DISTRICT, SHANGHAI (CN); LOTTA STENMAN, Kantvik (FI)

(21) Appl. No.: 17/311,155

(22) PCT Filed: Dec. 5, 2019
(86) PCT No.: PCT/EP2019/083816

§ 371 Date: Jun. 4, 2021

(51) Int. Cl. A61K 35/747; C12N 1/20; A61K 9/00; A61K 9/19; A61P 25/22; A61P 25/24; A23L 33/135; A23L 33/00; A23L 33/125; A23L 2/52

(52) U.S. Cl. CPC A61K 35/747; C12N 1/205; A61K 9/0053; A61K 9/19; A61P 25/22; C12R 2001/25; A23L 33/135; A23L 33/40; A23L 33/125; A23L 2/52; A61P 25/24

(57) ABSTRACT

The invention relates to compositions comprising Lactobacillus plantarum, and methods and uses of said compositions for preventing and/or treating mental illness, symptoms affecting mental health and/or conditions associated with chronic stress, in a mammal. The invention can be applied in restoring, maintaining, and/or promoting mental (including cognitive) health and has potential applications, inter alia, in the area of dietary and food supplements, medicaments and pharmaceuticals.

### FIELD OF THE INVENTION

This invention relates to a bacterial strain of the species Lactobacillus plantarum, particularly, but not exclusively, the bacterial strain LP12151. This invention also relates to compositions comprising Lactobacillus plantarum, particularly, but not exclusively, the bacterial strain LP12151. This invention also relates to compositions comprising Lactobacillus plantarum, particularly, but not exclusively, the bacterial strain LP12151 for use in preventing and/or treating mental illness, symptoms affecting mental health and/or conditions associated with chronic stress in a mammal. This invention also relates to methods and uses of bacterial strain of the species Lactobacillus plantarum and compositions comprising Lactobacillus plantarum, including food products, beverage, dietary supplements, and pharmaceutically acceptable formulations or compositions.

### BACKGROUND

Mental health is related to emotional, psychological, physical and social well-being. Our mental health status determines how we handle stress. A mental illness can be defined as a health condition that changes a person's thinking, feelings, or behaviour (or all three) and that causes the person distress and problems functioning in social, work or family activities. Mental illness encompasses a wide range of disorders related to anxiety, mood, psychosis, eating behaviour, impulse control and addiction, personality, sociability, dissociation, obsessive-compulsive and post-traumatic stress. Each illness alters a person's thoughts, feelings, and/or behaviours in distinct ways. Disorders such as Parkinson's disease, epilepsy and multiple sclerosis are brain disorders but they are considered neurological diseases rather than mental illness. Interestingly, the lines between mental illness and neurological diseases, including memory disorders such as mild cognitive impairment, dementia and Alzheimer's disease, are not clearly defined and increasing evidence now suggests that mental illness is associated with changes in the brain's structure, chemistry and function which could underlie the development of neurological disorders. For example, the link between neurocognitive deficits and mood disorders is well established such that in major depression, cognitive impairment can mimic that observed in dementia (Rabins et al. Br J Psychiatry 1984; 144: 488-92).

Furthermore, untreated chronic stress can result in serious health conditions such as anxiety, muscle pain, high blood pressure and a weakened immune system. Research shows that stress can contribute to the development of major illnesses, such as heart disease, depression and obesity. Symptoms of acute and chronic stress can manifest in the gastrointestinal tract, causing short- and long-term effects on the functions of the gastrointestinal tract, respectively. Exposure to stress results in alterations within the gut-brain axis, ultimately leading to the development of a broad array of gastrointestinal disorders including inflammatory bowel disease (IBD), irritable bowel syndrome (IBS) and other functional gastrointestinal diseases, food antigen-related adverse responses, peptic ulcers and gastroesophageal reflux disease (GERD). The major effects of stress on gut physiology include: 1) alterations in gastrointestinal motility; 2) increase in visceral perception; 3) changes in gastrointestinal secretion; 4) increase in intestinal permeability; 5) negative effects on regenerative capacity of gastrointestinal mucosa and mucosal blood flow; and 6) alteration in gut microbial composition (Konturek et al. J. Physiol Pharmacol. 2011; 62(6):591-9).

With respect to mental illness and associated neurocognitive decline and neurological disorders, there is now a clear emphasis on strategies to achieve positive mental and cognitive health for a full and healthy life. There is an increase in demand for nutritional therapies to achieve positive mental health, with no side effects. Current medication to treat mental illnesses symptoms affecting mental health have many negative side effects such as nausea, increased appetite and weight gain, fatigue and gastrointestinal symptoms. Dietary supplements may represent an attractive means of achieving positive mental health and preventing symptoms of mental illness and related conditions from developing.

The gut-brain axis describes the bidirectional communication that exists between the brain and the gut and the microbiota-gut-brain axis supports the role of the gut microbiome in this communication system. As outlined above, mental illness and symptoms affecting mental health are comorbid with gastrointestinal disorders whereby emotional and routine daily life stress can disrupt digestive function and vice versa. Increasing evidence indicates that the gut microbiota exerts a profound influence on brain physiology, psychological responses and ultimately behaviour (Dinan et al. J. Psychiatr Res. 2015; 63: 1-9). Emerging evidence suggests that probiotics can influence central nervous system function and regulate mood, psychological symptoms such as anxiety and depression and stress-related changes in physiology, behaviour and brain function.

### OBJECT OF INVENTION

It is an object of the present invention to provide means for preventing and/or treating a mental illness, a symptom affecting mental health and/or a condition associated with chronic stress. It is therefore an object of the invention to provide means by which an individual's mental health can be promoted, maintained, and/or restored.

The present invention is based on studies described herein which surprisingly demonstrate that Lactobacillus plantarum can significantly counteract the effects of stress on behavioural (anxiety and depression), biochemical and functional outcomes.

### SUMMARY OF THE INVENTION

In one aspect, the invention provides a bacterial strain of the species Lactobacillus plantarum, wherein the bacterial strain is strain LP12151 deposited with DSMZ on Jan. 9, 2018 under accession number DSM 32721.

In another aspect, the invention provides a bacteria of the species Lactobacillus plantarum or a mixture thereof for use in preventing and/or treating a mental illness, a symptom affecting mental health and/or a condition associated with chronic stress in a mammal.

In another aspect, the invention provides a bacteria of the species Lactobacillus plantarum or a mixture thereof for use in preventing and/or treating a mental illness, a symptom affecting mental health and/or a condition associated with chronic stress in a mammal, wherein the bacteria of the species Lactobacillus plantarum is strain LP12151 deposited with DSMZ on Jan. 9, 2018 under accession number DSM 32721.

In yet a further aspect, the invention provides a composition comprising a bacterial strain of the species Lactobacillus plantarum, wherein the bacterial strain is strain LP12151 deposited with DSMZ on Jan. 9, 2018 under accession number DSM 32721.

In another aspect, the invention provides a composition comprising a bacterium of the species Lactobacillus plantarum for use in preventing and/or treating a mental illness, a symptom affecting mental health and/or a condition associated with chronic stress in a mammal.

In another aspect, the invention provides a composition comprising a bacterium of the species Lactobacillus plantarum for use in preventing and/or treating a mental illness, a symptom affecting mental health and/or a condition associated with chronic stress in a mammal, wherein the strain of Lactobacillus plantarum is the strain LP12151 deposited with the DSMZ on Jan. 9, 2018 under accession number DSM 32721.

In another aspect, the invention provides a use of bacteria of the species Lactobacillus plantarum or a mixture thereof for preventing and/or treating a mental illness, a symptom affecting mental health and/or a condition associated with chronic stress in a mammal.

In a further aspect, the invention provides a use of a composition comprising a bacterium of the species Lactobacillus plantarum for preventing and/or treating a mental illness, a symptom affecting mental health and/or a condition associated with chronic stress in a mammal.

In yet a further aspect, the invention provides a method for preventing and/or treating a mental illness, a symptom affecting mental health and/or a condition associated with chronic stress in a mammal comprising administering to the mammal a bacteria of the species Lactobacillus plantarum or a mixture thereof, wherein the administration of the bacteria prevents and/or treats the mental illness, the symptom affecting mental health and/or the condition associated with chronic stress in the mammal.

In yet a further aspect, the invention provides a method for preventing and/or treating a mental illness, a symptom affecting mental health and/or a condition associated with chronic stress in a mammal comprising administering to the mammal a composition comprising a bacterium of the species Lactobacillus plantarum, wherein the administration of the composition prevents and/or treats the mental illness, the symptom affecting mental health and/or the condition associated with chronic stress in the mammal.

### DESCRIPTION OF DRAWINGS

FIG. 1. Effect of 3 weeks of chronic stress on body weight in mice, in groups treated with vehicle alone or a selected bacterial strain. N=12 for all groups except for stressed/vehicle group where N=11 and stressed/LP12418 group where N=11. Statistical Analyses: Two-way ANOVA; Weight (time): F_{(12,636)}=30.38, p<0.0001; Weight (treatment): F_{(4,636)}=6.54, p<0.0001

FIG. 2. Effect of treatment with selected bacterial strains on chronic stress-induced anxiety in mice in the open field procedure. Three parameters were measured; locomotor activity (A), locomotion in the centre of the area (B), and rearing/grooming behaviour [stereotypies (C)]. For stereotypies, data were represented as arbitrary units (A.U.) corresponding to the number of rearings plus the number of groomings during 10 minutes of open field procedure. N=12 for all groups except for stressed/vehicle group where N=11 and stressed/LP12418 group where N=11. Statistical analyses: One-way ANOVA; Locomotor activity: F_{(4,57)}=0.4278, p>35 0.05; Locomotion in the centre: F_{(4,57)}=58.20, p<0.0001; Stereotypes: F_{(4,57)}=0.3330, p>0.05; Pairwise comparisons: ***p<0.001 vs. the non-stress/vehicle group, ###p<0.001 vs the chronic stress/vehicle group (Dunnett's test)

FIG. 3. Effect of treatment with selected bacterial strains on chronic stress-induced behavioural despair in mice as measured in the forced swim test. Three parameters were measured; swim time (A), struggle time (B) and immobility time (C). N=12 for all groups. Statistical analyses: One-way ANOVA; Swim time: F_{(4,57)}=7.596 p<0.0001; Struggle time: F_{(4,57)}=0.8225, p>0.05; Immobility time: F_{(4,57)}=28.10, p<0.0001; Pairwise comparisons: **p<0.01, ***p<0.001 vs the non-stressed/vehicle group, ##p<0.01, ###p<0.001 vs the chronic stress/vehicle group (Dunnett's test).

### DETAILED DESCRIPTION OF INVENTION

### Bacteria and Deposit

The bacteria used in aspects of the invention are bacteria of the species Lactobacillus plantarum. Novel Lactobacillus plantarum strain of the invention is strain LP12151.

Lactobacillus plantarum strain LP12151 (DGCC12151) was deposited with the DSMZ (Leibniz-Institut DSMZ-Deutsche Sammlung von Mikroorganismen und Zellkulturen GmbH, Inhoffenstrasse 7B, D-38124 Braunschweig—Germany) on Jan. 9, 2018 under accession number DSM 32721.

Other bacteria used in aspects of the invention are:

Lactobacillus plantarum strain LP12418 (DGCC12418) was deposited with the DSMZ (Leibniz-Institut DSMZ-Deutsche Sammlung von Mikroorganismen und Zellkulturen GmbH, Inhoffenstrasse 7B, D-38124 Braunschweig—Germany) on Sep. 27, 2017 under accession number DSM 32655.

Lactobacillus plantarum strain LP12407 (DGCC12407) was deposited with the DSMZ (Leibniz-Institut DSMZ-Deutsche Sammlung von Mikroorganismen und Zellkulturen GmbH, Inhoffenstrasse 7B, D-38124 Braunschweig—Germany) on Sep. 27, 2017 under accession number DSM 32654.

Lactobacillus paracasei strain Lpc-37 (DGCC4981) was deposited with the DSMZ (Leibniz-Institut DSMZ-Deutsche Sammlung von Mikroorganismen und Zellkulturen GmbH, Inhoffenstrasse 7B, D-38124 Braunschweig—Germany) on Oct. 5, 2017 under accession number DSM 32661.

In one aspect, the present invention relates to a bacterial strain of the species Lactobacillus plantarum. More particularly, the present invention relates to the bacterial strain LP12151 deposited with DSMZ on Jan. 9, 2018 under accession number DSM 32721.

In another aspect, the present invention relates Bacteria of the species Lactobacillus plantarum or a mixture thereof for use in preventing and/or treating a mental illness, a symptom affecting mental health and/or a condition associated with chronic stress in a mammal.

In a further aspect, the present invention relates to a use of bacteria of the species Lactobacillus plantarum or a mixture thereof for preventing and/or treating a mental illness, a symptom affecting mental health and/or a condition associated with chronic stress in a mammal.

In a yet further aspect, the present invention relates a method for preventing and/or treating a mental illness, a symptom affecting mental health and/or a condition associated with chronic stress in a mammal comprising administering to the mammal a bacteria of the species Lactobacillus plantarum or a mixture thereof, wherein the administration of the bacteria prevents and/or treats the mental illness, the symptom affecting mental health and/or the condition associated with chronic stress in the mammal.

In another aspect, the present invention relates to a method for preventing and/or treating a mental illness, a symptom affecting mental health and/or a condition associated with chronic stress in a mammal comprising administering to the mammal a composition comprising a bacterium of the species Lactobacillus plantarum, wherein the administration of the composition prevents and/or treats the mental illness, the symptom affecting mental health and/or the condition associated with chronic stress in the mammal.

According to one aspect of the present invention, the mental illness is a mood disorder, an anxiety disorder and/or depression.

According to another aspect of the present invention, the symptom affecting mental health is anxiety, mood swings disorder and/or depression.

According to another aspect of the present invention, the condition associated with chronic stress is a gastrointestinal disorder, e.g. irritable bowel syndrome.

In a further aspect of the present invention, the bacteria of the species Lactobacillus plantarum is a probiotic Lactobacillus plantarum or a mixture thereof.

The term “probiotic bacteria” is defined as covering any non-pathogenic bacteria which, when administered live in adequate amounts to a host, confer a health benefit on that host. For classification as a “probiotic”, the bacteria must survive passage through the upper part of the digestive tract of the host. They are non-pathogenic, non-toxic and exercise their beneficial effect on health on the one hand via ecological interactions with the resident flora in the digestive tract, and on the other hand via their ability to influence the host physiology and immune system in a positive manner. Probiotic bacteria, when administered to a host in sufficient number, have the ability to progress through the intestine, maintaining viability, exerting their primary effects in the lumen and/or the wall of the host's gastrointestinal tract. They then transiently form part of the resident flora and this colonisation (or transient colonisation) allows the probiotic bacteria to exercise a beneficial effect, such as the repression of potentially pathogenic micro-organisms present in the flora and interactions with the host in the intestine including the immune system.

In one aspect of the invention, the bacteria of the species Lactobacillus plantarum or a mixture thereof for use in preventing and/or treating a mental illness, a symptom affecting mental health and/or a condition associated with chronic stress in a mammal is strain LP12151 deposited with DSMZ on Jan. 9, 2018 under accession number DSM 32721.

In another aspect of the present invention, the bacteria of the species Lactobacillus plantarum or a mixture thereof used for preventing and/or treating a mental illness, a symptom affecting mental health and/or a condition associated with chronic stress in a mammal is strain LP12151 deposited with DSMZ on Jan. 9, 2018 under accession number DSM 32721.

In a further aspect of the present invention, the bacteria used in the method for preventing and/or treating a mental illness, a symptom affecting mental health and/or a condition associated with chronic stress in a mammal is strain LP12151 deposited with DSMZ on Jan. 9, 2018 under accession number DSM 32721.

The Lactobacillus plantarum strain LP12151 may also be used in combination with one or more other bacterial species which have the ability to exert positive health benefits on a host to which they are administered.

Optionally the Lactobacillus plantarum LP12151 strain of the invention are used in combination with other probiotic bacteria, e.g. Lactobacillus paracasei strain Lpc-37.

The Lactobacillus plantarum strain LP12151 may be used in any form (for example viable, dormant, inactivated or dead bacteria), provided that the bacteria remain capable of exerting the effects described herein. Preferably, the Lactobacillus plantarum strain LP12151 used in aspects of the invention is viable.

The Lactobacillus plantarum strain LP12151, when used in aspects of the invention, is suitable for human and/or animal consumption. A skilled person will be readily aware of specific strains of Lactobacillus plantarum which are used in the food and/or agricultural industries and which are generally considered suitable for human and/or animal consumption.

The invention further provides a mutant, a variant and/or a progeny of the deposited bacterial strain.

As used herein, the term “mutant” refers to any microorganism resulting from modification of the parent (i.e. deposited) strain. For example, a mutant may be a microorganism resulting from genetically modifying a deposited strain.

As used herein, the term “variant” refers to a naturally occurring microorganism which is derived from the parent (i.e. deposited strain). For example, a variant may be a microorganism resulting from adaption to particular cell culture conditions.

As used herein, the term “progeny” means any microorganism resulting from the reproduction or multiplication of anyone of the deposited strains. Therefore, “progeny” means any direct descendant of anyone of the deposited strains. As such, the progeny strain may itself be identified as the same strain as the parent (i.e. deposited) strain. It will be apparent to one skilled in the art that due to the process of asexual reproduction, a progeny strain will be genetically virtually identical to the parent strain. Accordingly, in one embodiment, the progeny may be genetically identical to the parent strain and may be considered to be a “clone” of the parent strain. Alternatively, the progeny may be substantially genetically identical to the parent strain.

The mutant, variant or progeny may have at least 90, 95, 98, 99, 99.5 or 99.9% sequence identity over the entire length of the bacterial genome with their parent strain. Furthermore, the mutant, variant or progeny will retain the same phenotype as the deposited parent strain.

### Compositions

The term “composition” is used in the broad sense to mean the manner in which something is composed, i.e. its general makeup.

In one aspect of the invention, the compositions may consist essentially of a single strain of Lactobacillus plantarum bacteria (e.g. LP12151/DGCC12151/DSM 32721).

In one aspect, the present invention relates to a composition comprising a bacterial strain of the species Lactobacillus plantarum, wherein the bacterial strain is strain LP12151 deposited with DSMZ on Jan. 9, 2018 under accession number DSM 32721.

In another aspect, the present invention relates to a composition comprising a bacterium of the species Lactobacillus plantarum for use in preventing and/or treating a mental illness, a symptom affecting mental health and/or a condition associated with chronic stress in a mammal.

The present invention also relates to a use of a composition comprising a bacterium of the species Lactobacillus plantarum for preventing and/or treating a mental illness, a symptom affecting mental health and/or a condition associated with chronic stress in a mammal.

The present invention further relates to a method for preventing and/or treating a mental illness, a symptom affecting mental health and/or a condition associated with chronic stress in a mammal comprising administering to the mammal a composition comprising a bacterium of the species Lactobacillus plantarum, wherein the administration of the composition prevents and/or treats the mental illness, the symptom affecting mental health and/or the condition associated with chronic stress in the mammal.

The bacterium of the species Lactobacillus plantarum comprised in the composition used in the method for preventing and/or treating a mental illness, a symptom affecting mental health and/or a condition associated with chronic stress in a mammal is strain LP12151 deposited with the DSMZ on Jan. 9, 2018 under accession number DSM 32721.

Alternatively, the compositions of the invention may additionally comprise other components, such as other bacterial strains (e.g. Lactobacillus paracasei strain Lpc-37 registered at the DSMZ under the deposit number DSM 32661), biological and chemical components, active ingredients, metabolites, nutrients, fibres, prebiotics, etc. While it is not a requirement that the compositions of the invention comprise any support, diluent or excipient, such a support, diluent or excipient may be added and used in a manner which is familiar to those skilled in the art. Examples of suitable excipients include, but are not limited to, microcrystalline cellulose, rice maltodextrin, silicone dioxide, and magnesium stearate.

Therefore, the present invention also relates to a composition which further comprises another bacteria strain.

The present invention also relates to compositions comprising prebiotics. A particular example of a prebiotics is polydextrose.

In another aspect, the present invention relates to compositions which are spray dried or freeze-dried compositions.

The compositions of the present invention may also comprise cryoprotectant components, for example, glucose, sucrose, lactose, trehalose, sodium ascorbate and/or other suitable cryoprotectants.

According to one aspect of the invention, the mental illness to be prevented and or treated is a mood disorder, an anxiety disorder and/or depression.

In another aspect of the invention, the symptom affecting mental health is anxiety, mood swings disorder and/or depression.

In a further aspect of the invention, the condition associated with chronic stress is a gastrointestinal disorder, e.g. irritable bowel syndrome.

In the present invention, the terms “composition” and “formulation” may be used interchangeably.

Compositions used in aspects of the invention may take the form of solid, solution or suspension preparations. Examples of solid preparations include, but are not limited to: tablets, pills, capsules, granules and powders which may be wettable, spray-dried or freeze dried/lyophilized. The compositions may contain flavouring or colouring agents. The compositions may be formulated for immediate-, delayed-, modified-, sustained-, pulsed- or controlled-release applications.

By way of example, if the compositions of the present invention are used in a tablet form, the tablets may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycollate, croscarmellose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc may be included.

Examples of other acceptable carriers for use in preparing compositions include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, hydroxymethylceilulose, polyvinylpyrrolidone, and the like.

For aqueous suspensions and/or elixirs, the compositions of the present invention may be combined with various sweetening or flavouring agents, colouring matter or dyes, with emulsifying and/or suspending agents and with diluents such as water, propylene glycol and glycerin, and combinations thereof.

Specific non-limiting examples of compositions which can be used in aspects of the invention are set out below for illustrative purposes. These include, but are not limited to food products, functional foods, dietary supplements, pharmaceutical compositions and medicaments.

### Dietary Supplements

The bacteria of the species Lactobacillus plantarum, for example strain LP12151, and the compositions of the present invention may take the form of dietary supplements or may themselves be used in combination with dietary supplements, also referred to herein as food supplements.

The term “dietary supplement” as used herein refers to a product intended for ingestion that contains a “dietary ingredient” intended to add nutritional value or health benefits to (supplement) the diet. A “dietary ingredient” may include (but is not limited to) one, or any combination, of the following substances: bacteria, a probiotic (e.g. probiotic bacteria), a vitamin, a mineral, a herb or other botanical, an amino acid, a dietary substance for use by people to supplement the diet by increasing the total dietary intake, a concentrate, metabolite, constituent, or extract.

Dietary supplements may be found in many forms such as tablets, capsules, soft gels, gel caps, liquids, or powders. Some dietary supplements can help ensure an adequate dietary intake of essential nutrients; others may help reduce risk of disease.

In one aspect, the present invention relates to a composition in the form of a food product, a beverage, a dietary supplement or a pharmaceutically acceptable formulation or composition.

### Food Products

The bacteria of the species Lactobacillus plantarum, for example strain LP12151, and/or the compositions of the invention may take the form of a food product. Here, the term “food” is used in a broad sense and covers food and drink for humans as well as food and drink for animals (i.e. a feed). Preferably, the food product is suitable for, and designed for, human consumption.

The food may be in the form of a liquid, solid or suspension, depending on the use and/or the mode of application and/or the mode of administration.

When in the form of a food product, the composition may comprise or be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the compositions of the invention may take the form of one of the following:

A fruit juice; a beverage comprising whey protein: a health or herbal tea, a cocoa drink, a milk drink, a lactic acid bacteria drink, a yoghurt and/or a drinking yoghurt, a cheese, an ice cream, a water ice, a desserts, a confectionery, a biscuit, a cake, cake mix or cake filling, a snack food, a fruit filling, a cake or doughnut icing, an instant bakery filling cream, a filling for cookies, a ready-to-use bakery filling, a reduced calorie filling, an adult nutritional beverage, an acidified soy/juice beverage, a nutritional or health bar, a beverage powder, a calcium fortified soy milk, or a calcium fortified coffee beverage.

Optionally, where the product is a food product, the Lactobacillus plantarum, for example strain LP12151, should remain effective through the normal “sell-by” or “expiration” date during which the food product is offered for sale by the retailer. Preferably, the effective time should extend past such dates until the end of the normal freshness period when food spoilage becomes apparent. The desired lengths of time and normal shelf life will vary from foodstuff to foodstuff and those of ordinary skill in the art will recognise that shelf-life times will vary upon the type of foodstuff, the size of the foodstuff, storage temperatures, processing conditions, packaging material and packaging equipment.

### Food Ingredients

The bacteria of the species Lactobacillus plantarum, for example strain LP12151, and/or compositions of the present invention may take the form of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

### Functional Foods

The bacteria of the species Lactobacillus plantarum, for example strain LP12151, and/or compositions of the invention may take the form of functional foods.

As used herein, the term “functional food” means food which is capable of providing not only a nutritional effect but is also capable of delivering a further beneficial effect to the consumer.

Accordingly, functional foods are ordinary foods that have components or ingredients (such as those described herein) incorporated into them that impart to the food a specific function—e.g. medical or physiological benefit—other than a purely nutritional effect.

Although there is no legal definition of a functional food, most of the parties with an interest in this area agree that they are foods marketed as having specific health effects beyond basic nutritional effects.

Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

### Medical Foods

The bacteria of the species Lactobacillus plantarum, for example strain LP12151, and/or compositions of the present invention may take the form of medical foods.

By “medical food” it is meant a food which is formulated to be consumed or administered with or without the supervision of a physician and which is intended for a specific dietary management or condition for which distinctive nutritional requirements, based on recognized scientific principles, are established by medical evaluation.

### Pharmaceutical Compositions

The bacteria of the species Lactobacillus plantarum, for example strain LP12151, and/or compositions of the invention may be used as—or in the preparation of—pharmaceuticals. Here, the term “pharmaceutical” is used in a broad sense—and covers pharmaceuticals for humans as well as pharmaceuticals for animals (i.e. veterinary applications). In a preferred aspect, the pharmaceutical is for human use.

The pharmaceutical can be for therapeutic purposes—which may be curative or palliative or preventative in nature.

A pharmaceutical may be in the form of a compressed tablet, tablet, capsule, ointment, suppository or drinkable solution.

When used as—or in the preparation of—a pharmaceutical, the bacteria of the species Lactobacillus plantarum, for example strain LP12151 and/or the compositions of the present invention may be used in conjunction with one or more of: a pharmaceutically acceptable carrier, a pharmaceutically acceptable diluent, a pharmaceutically acceptable excipient, a pharmaceutically acceptable adjuvant, a pharmaceutically active ingredient.

The pharmaceutical may be in the form of a liquid or as a solid—depending on the use and/or the mode of application and/or the mode of administration.

The Lactobacillus plantarum strain used in the present invention may constitute the pharmaceutically active ingredient or ingredients. In one embodiment, Lactobacillus plantarum LP12151 constitutes the sole active ingredient. Alternatively, LP12151 may be at least one of a number (i.e. 2 or more) of pharmaceutically active ingredients.

### Medicaments

The bacteria of the species Lactobacillus plantarum, for example strain LP12151, and/or Compositions of the invention may take the form of medicaments.

The term “medicament” as used herein encompasses medicaments for both human and animal usage in human and veterinary medicine. In addition, the term “medicament” as used herein means any substance which provides a therapeutic, preventative and/or beneficial effect. The term “medicament” as used herein is not necessarily limited to substances which need Marketing Approval but may include substances which can be used in cosmetics, nutraceuticals, food (including feeds and beverages for example), probiotic cultures, and natural remedies. In addition, the term “medicament” as used herein encompasses a product designed for incorporation in animal feed, for example livestock feed and/or pet food.

### Dosage

The compositions of the present invention may comprise from 10⁶ to 10¹² colony forming units (CFU) of Lactobacillus plantarum bacteria per dose, per unit dose or per gram of composition, and more particularly from 10⁸ to 10¹² CFU per dose, per unit dose or per gram of composition. Optionally the compositions comprise about 10¹⁰ CFU Lactobacillus plantarum per dose, per unit dose or per gram of composition.

The Lactobacillus plantarum LP12151 strain of the invention may be administered at a dosage of from about 10⁶ to about 10¹² CFU of bacteria per unit dose, preferably about 10⁸ to about 10¹² CFU of bacteria per dose. By the term “per dose” it is meant that this amount of bacteria is provided to a subject either per day or per intake, preferably per day. For example, if the bacteria are to be administered in a food product, for example in a yoghurt, then the yoghurt may contain from about 10⁶ to 10¹² CFU of LP12151. Alternatively, however, this amount of bacteria may be split into multiple administrations, each consisting of a smaller amount of microbial loading—so long as the overall amount of Lactobacillus plantarum received by the subject in any specific time, for instance each 24-hour period, is from about 10⁶ to about 10¹² CFU of bacteria, optionally 10⁸ to about 10¹² CFU of bacteria.

In accordance with the present invention an effective amount of LP12151 may be at least 10⁶ CFU of bacteria/dose, optionally from about 10⁸ to about 10¹² CFU of bacteria/dose, e.g., about 10¹⁰ CFU of bacteria/dose.

In one embodiment, the LP12151 may be administered at a dosage of from about 10⁶ to about 10¹² CFU of bacteria/day, optionally about 10⁸ to about 10¹² CFU of bacteria/day. Hence, the effective amount in this embodiment may be from about 10⁶ to about 10¹² CFU of bacteria/day, optionally about 10⁸ to about 10¹² CFU of bacteria/day.

### Effects/Subjects/Medical Indications

The compositions of the present invention can be used for administration to a mammal, including for example livestock (including cattle, horses, pigs, and sheep), and humans. In some embodiments of the present invention, the mammal is a companion animal (including pets), such as a dog or a cat for instance. In preferred embodiments, the compositions are for use in a human.

The compositions of the present invention can be used for the prevention and/or treatment of a mental illness, a symptom affecting mental health and/or a condition associated with chronic stress, such as for example, neurological and gastrointestinal disorders.

The term “mental illness” can be defined as a health condition that changes a person's thinking, feelings, or behaviour (or all three) and that causes the person distress and problems functioning in social, work or family activities. Mental illness encompasses a wide range of disorders related to anxiety, mood, psychosis, eating behaviour, impulse control and addiction, personality, sociability, dissociation, obsessive-compulsive and post-traumatic stress. Each illness alters a person's thoughts, feelings, and/or behaviours in distinct ways. As used herein, mental illness also includes neurological disorders and conditions related to mental illness which may be a cause or symptom of a mental illness or be a condition that can increase the chance of one developing.

Disorders associated with anxiety are categorised under “mental illness”. The term “anxiety disorder” refers to a specific mental illness that involves extreme fear or worry, and includes generalized anxiety disorder (GAD), panic disorder and panic attacks, agoraphobia, social anxiety disorder, selective mutism, separation anxiety, and specific phobias. Obsessive-compulsive disorder (OCD) and posttraumatic stress disorder (PTSD) are closely related to anxiety disorders, which some may experience at the same time as depression. GAD represents more than the normal level of anxiety individuals experience from day to day and is characterised by chronic worry and tension. Compositions of the invention can be used to treat and/or prevent recognised anxiety disorders as well as symptoms of anxiety more generally.

As used herein, mental illness also includes associated neurological disorders, including memory disorders, mild cognitive impairment, dementia and Alzheimer's disease. Cognition denotes a relatively high level of processing of specific information including thinking, memory, perception, motivation, skilled movements and language. Cognitive disorders are defined as those with “a significant impairment of cognition or memory that represents a marked deterioration from a previous level of function” (Guerrero, Anthony (2008), “Problem-Based Behavioral Science of Medicine”; New York: Springer. pp. 367-79). They can be categorised into three main areas: (1) Delirium, a disorder affecting situational awareness and processing of new information; (2) Dementia, a disorder which can erase all or parts of an individual's memory; and (3) Amnesia, a disorder in which the individual afflicted has trouble retaining long term memories.

The compositions of the invention can be used to promote, restore and/or maintain an individual's mental health, such as to prevent mental illness or any associated disorders and/or symptoms affecting an individual's mental health.

Symptoms affecting mental health include; feeling sad or down, confused thinking or reduced ability to concentrate, excessive fears or worries, or extreme feelings of guilt, extreme mood changes of highs and lows, withdrawal from friends and activities, detachment from reality, paranoia or hallucinations, inability to cope with daily problems or stress, trouble understanding and relating to situations and to people, alcohol or drug abuse, major changes in eating habits, sex drive changes, excessive anger, hostility or violence and suicidal thoughts.

For the purposes of the present invention, mental illness and symptoms affecting mental health, also encompass conditions affecting an individual's cognitive function. Such conditions may include or overlap with various cognitive disorders. Examples include, but are not limited to, agnosia, amnesia, dementia, Alzheimer's disease, Parkinson's disease, and chronic stress, which has been shown to negatively affect brain function.

Intense acute and chronic stress can negatively impact both physical and mental health, increasing risk of developing mental illness. For example, chronic stress has been correlated with the development of mood disorders, anxiety disorders and depression. The compositions of the invention can be used to prevent and/or treat a mental illness or symptoms affecting mental health, resulting from chronic or acute stress.

The compositions of the invention can also be used to treat and/or prevent other (including physical) conditions associated with chronic or acute stress. For example, in one embodiment, the compositions of the invention are used to treat and/or prevent gastrointestinal disorders, for example, IBS, associated with chronic or acute stress. By addressing the symptoms of mental illness associated with gastrointestinal disorders, it is possible that such treatment may have a beneficial effect on the gastrointestinal disorders themselves.

More generally, the compositions of the invention can be used for the prevention and/or treatment of one or more of the mental illnesses, symptoms affecting mental health and/or conditions associated with chronic or intense acute stress as set out above.

In particular embodiments, the compositions of the invention can be used for the prevention and/or treatment of anxiety, depression, and/or diminished cognitive function.

When compositions of the invention are used for the prevention of a mental illness or a symptom affecting mental health, they can be used for maintaining a normal level of mental health in an already healthy individual. Alternatively, when compositions of the invention are used to treat a mental illness or symptom affecting mental health, they can be used for restoring or partially restoring a normal level of mental health in an individual suffering from the mental illness or symptom in question.

### Methods and Other Embodiments of the Invention

As set out above, one aspect of the invention provides a method for preventing and/or treating a mental illness, a symptom affecting mental health or a condition associated with chronic stress, in a mammal, comprising administering to the mammal a Lactobacillus plantarum or a composition comprising Lactobacillus plantarum.

In yet another aspect, the intervention provides for the use of Lactobacillus plantarum or a composition comprising Lactobacillus plantarum for the manufacture of a medicament for preventing and/or treating a mental illness, a symptom affecting mental health and or a condition associated with chronic stress, in a mammal.

For the avoidance of doubt, the bacteria and any of the compositions described herein and set out above can be utilised in the methods of the invention. Further embodiments include, but are not limited to, those set out below:

Embodiment 1. A bacterial strain of the species Lactobacillus plantarum, wherein the bacterial strain is strain LP12151 deposited with DSMZ on Jan. 9, 2018 under accession number DSM 32721.

Embodiment 2. Bacteria of the species Lactobacillus plantarum or a mixture thereof for use in preventing and/or treating a mental illness, a symptom affecting mental health and/or a condition associated with chronic stress in a mammal.

Embodiment 3. The bacteria for use according to embodiment 2, wherein the mental illness is a mood disorder, an anxiety disorder and/or depression.

Embodiment 4. The bacteria for use according to embodiment 2, wherein the symptom affecting mental health is anxiety, mood swings and/or depression

Embodiment 5. The bacteria for use according to embodiment 2, wherein the condition associated with chronic stress is a gastrointestinal disorder, e.g. irritable bowel syndrome.

Embodiment 6. The bacteria according to embodiment 2, wherein the bacteria of the species Lactobacillus plantarum is a probiotic Lactobacillus plantarum or a mixture thereof.

Embodiment 7. The bacteria according to any one of the embodiments 2 to 6, wherein the bacteria of the species Lactobacillus plantarum is strain LP12151 deposited with DSMZ on Jan. 9, 2018 under accession number DSM 32721.

Embodiment 8. A composition comprising a bacterial strain of the species Lactobacillus plantarum, wherein the bacterial strain is strain LP12151 deposited with DSMZ on January 9th, 2018 under accession number DSM 32721.

Embodiment 9. A composition comprising a bacterium of the species Lactobacillus plantarum for use in preventing and/or treating a mental illness, a symptom affecting mental health and/or a condition associated with chronic stress in a mammal.

Embodiment 10. The composition for use according to embodiment 9, wherein the strain of Lactobacillus plantarum is the strain LP12151 deposited with the DSMZ on Jan. 9, 2018 under accession number DSM 32721.

Embodiment 11. The composition according to any one of embodiments 8-10, wherein the composition further comprises another bacterial strain.

Embodiment 12. The composition according to any one of embodiments 8 to 12, wherein said composition further comprises prebiotics.

Embodiment 13. The composition according to embodiment 12, wherein the prebiotics is polydextrose.

Embodiment 14. The composition according to any one of embodiments 8-13, wherein the composition is in the form of a food product, a beverage, a dietary supplement or a pharmaceutically acceptable formulation or composition.

Embodiment 15. The composition according any one of the embodiments 8-14, wherein the composition is a spray dried or freeze-dried composition.

Embodiment 16. The composition according to embodiment 15, wherein the composition comprises a cryoprotectant.

Embodiment 17. The composition according to any one of embodiments 8-16, wherein the composition is in unit dosage form and wherein the Lactobacillus plantarum strain is present in the composition in an amount between 10⁶ and 10¹² CFU, e.g. between 10⁸ and 10¹² CFU, optionally 10¹⁰ CFU per unit dose.

Embodiment 18. The composition according to embodiment 9, wherein the mental illness is a mood disorder, an anxiety disorder and/or depression.

Embodiment 19. The composition according to embodiment 9, wherein the symptom affecting mental health is anxiety, mood swings and/or depression.

Embodiment 20. The composition according to embodiment 9, wherein the condition associated with chronic stress is a gastrointestinal disorder, e.g. irritable bowel syndrome.

Embodiment 21. Use of bacteria of the species Lactobacillus plantarum or a mixture thereof for preventing and/or treating a mental illness, a symptom affecting mental health and/or a condition associated with chronic stress in a mammal.

Embodiment 22. The use according to embodiment 21, wherein the mental illness is a mood disorder, an anxiety disorder and/or depression.

Embodiment 23. The use according to claim 21, wherein the symptom affecting mental health is anxiety, mood swings and/or depression

Embodiment 24. The use according to embodiment 21, wherein the condition associated with chronic stress is a gastrointestinal disorder, e.g. irritable bowel syndrome.

Embodiment 25. The use according to embodiment 21, wherein the bacteria of the species Lactobacillus plantarum is a probiotic Lactobacillus plantarum or a mixture thereof.

Embodiment 26. The use according to any one of the embodiments 21 to 25, wherein the bacteria of the species Lactobacillus plantarum is strain LP12151 deposited with DSMZ on Jan. 9, 2018 under accession number DSM 32721.

Embodiment 27. Use of a composition comprising a bacterium of the species Lactobacillus plantarum for preventing and/or treating a mental illness, a symptom affecting mental health and/or a condition associated with chronic stress in a mammal.

Embodiment 28. The use according to embodiment 27, wherein the strain of Lactobacillus plantarum is the strain LP12151 deposited with the DSMZ on Jan. 9, 2018 under accession number DSM 32721.

Embodiment 29. The use according to any one of embodiments 27-28, wherein the composition further comprises another bacterial strain.

Embodiment 30. The use according to any one of embodiments 27 to 29, wherein said composition further comprises prebiotics.

Embodiment 31. The use according to embodiment 30, wherein the prebiotics is polydextrose.

Embodiment 32. The use according to any one of embodiments 27-31, wherein the composition is in the form of a food product, a beverage, a dietary supplement or a pharmaceutically acceptable formulation or composition.

Embodiment 33. The use according any one of the embodiments 27-32, wherein the composition is a spray dried or freeze-dried composition.

Embodiment 34. The use according to embodiment 33, wherein the composition comprises a cryoprotectant.

Embodiment 35. The use according to any one of embodiments 27-34, wherein the composition is in unit dosage form and wherein the Lactobacillus plantarum strain is present in the composition in an amount between 10⁶ and 10¹² CFU, e.g. between 10⁸ and 10¹² CFU, optionally 10¹⁰ CFU per unit dose.

Embodiment 36. The use according to embodiment 27, wherein the mental illness is a mood disorder, an anxiety disorder and/or depression.

Embodiment 37. The use according to embodiment 27, wherein the symptom affecting mental health is anxiety, mood swings and/or depression.

Embodiment 38. The use according to embodiment 27, wherein the condition associated with chronic stress is a gastrointestinal disorder, e.g. irritable bowel syndrome.

Embodiment 39. Method for preventing and/or treating a mental illness, a symptom affecting mental health and/or a condition associated with chronic stress in a mammal comprising administering to the mammal a bacteria of the species Lactobacillus plantarum or a mixture thereof, wherein the administration of the bacteria prevents and/or treats the mental illness, the symptom affecting mental health and/or the condition associated with chronic stress in the mammal.

Embodiment 40. The method according to embodiment 39, wherein the mental illness is a mood disorder, an anxiety disorder and/or depression.

Embodiment 41. The method according to claim 39, wherein the symptom affecting mental health is anxiety, mood swings and/or depression

Embodiment 42. The use according to embodiment 39, wherein the condition associated with chronic stress is a gastrointestinal disorder, e.g. irritable bowel syndrome.

Embodiment 43. The method according to embodiment 39, wherein the bacteria of the species Lactobacillus plantarum is a probiotic Lactobacillus plantarum or a mixture thereof.

Embodiment 44. The method according to any one of the embodiments 39 to 43, wherein the bacteria of the species Lactobacillus plantarum is strain LP12151 deposited with DSMZ on Jan. 9, 2018 under accession number DSM 32721.

Embodiment 45. Method for preventing and/or treating a mental illness, a symptom affecting mental health and/or a condition associated with chronic stress in a mammal comprising administering to the mammal a composition comprising a bacterium of the species Lactobacillus plantarum, wherein the administration of the composition prevents and/or treats the mental illness, the symptom affecting mental health and/or the condition associated with chronic stress in the mammal.

Embodiment 46. The method according to embodiment 45, wherein the strain of Lactobacillus plantarum is the strain LP12151 deposited with the DSMZ on Jan. 9, 2018 under accession number DSM 32721.

Embodiment 47. The method according to any one of embodiments 45-46, wherein the composition further comprises another bacterial strain.

Embodiment 48. The method according to any one of embodiments 45 to 47, wherein said composition further comprises prebiotics.

Embodiment 49. The method according to embodiment 48, wherein the prebiotics is polydextrose.

Embodiment 50. The method according to any one of embodiments 45-49, wherein the composition is in the form of a food product, a beverage, a dietary supplement or a pharmaceutically acceptable formulation or composition.

Embodiment 51. The method according any one of the embodiments 45-50, wherein the composition is a spray dried or freeze-dried composition.

Embodiment 52. The method according to embodiment 51, wherein the composition comprises a cryoprotectant.

Embodiment 53. The method according to any one of embodiments 45-52, wherein the composition is in unit dosage form and wherein the Lactobacillus plantarum strain is present in the composition in an amount between 10⁶ and 10¹² CFU, e.g. between 10⁸ and 10¹² CFU, optionally 10¹⁰ CFU per unit dose.

Embodiment 54. The method according to embodiment 45, wherein the mental illness is a mood disorder, an anxiety disorder and/or depression.

Embodiment 55. The method according to embodiment 45, wherein the symptom affecting mental health is anxiety, mood swings and/or depression.

Embodiment 56. The method according to embodiment 45, wherein the condition associated with chronic stress is a gastrointestinal disorder, e.g. irritable bowel syndrome.

### EXAMPLES

The following examples are provided in order to demonstrate and further illustrate specific embodiments and aspects of the present invention and are not to be construed as limiting the scope thereof.

### Example 1: Initial Screening of Candidate Strains

Multiple probiotic candidates were initially screened for probiotic characteristics, safety and manufacturing performance. Most of these candidates were discarded due to poor performance. Efficacy testing was conducted on the remaining candidates and from these, 3 strains were selected for further experimental testing, as reported in the examples below.

### Example 2: Materials and Methods—General

### Animals

Male Swiss mice, 5 weeks old and weighing 30-35 g, from JANVIER (Saint Berthevin, France), were used, and experiments took place at Amylgen (Direction Régionale de l'Alimentation, de l'Agriculture et de la Forêt du Languedoc-Roussillon). Animals were housed in groups of six mice with access to food and water ad libitum, except during behavioural experiments. Each cage contained mice from a single treatment group. They were kept in a temperature and humidity controlled animal facility on a 12-hour light/dark cycle (lights off at 07:00 pm). All animal procedures were conducted in strict adherence to the European Union directive of Sep. 22, 2010 (2010/63/UE).

### Bacterial Strain Formulation and Administration

The bacterial strains were solubilized in 0.9% NaCl and administered orally by gavage (100 μL per mouse) at 9:00 a.m. each morning, corresponding to a dose of 10⁹ CFU/day per mouse. Vehicle mice received 0.9% NaCl without bacterial strains. The duration of the treatment was 33 days in total.

### Chronic Stress Procedure

The chronic stress procedure was carried out as previously described (Espallergues et al., Psychoneuroendocrinology, 2009). Mice were repeatedly placed in Plexiglas transparent restraint tubes (12 cm length, 3 cm diameter) under bright light for a period of 180 min per day (11:00 a.m. to 2:00 p.m.), for 5 consecutive days per week, over three weeks. Control group animals (No stress mice/Vehicle) were never placed in restraint tubes and remained undisturbed during the stress procedure in a room different from the one where stress was taking place.

### Randomisation of the Animals

In each cage (N=6), animals received the same treatment. Treatments and stress procedure were performed in a random manner by an experimenter not involved in the behavioural and biochemical experiments. The behavioural procedures were conducted by a second different experimenter.

### Schedule of Experimental Procedures

On day 01, animals were randomly assigned to an experimental group, weighed and treated with the appropriate strain or vehicle.
From day 01 to day 33, animals were treated with appropriate strain or vehicle.
From day 08 to day 28, animals were submitted to chronic stress, 3 hours per day, five days per week from Monday to Friday, in accordance with the chronic stress procedure described above.
On day 29, animals performed the elevated plus maze procedure.
On day 30, animals performed the open field procedure, which was also habituation to the day 31-32 tasks.
From day 30 to day 32, animals performed the object recognition memory task.
On day 33, animals performed the forced swimming test.
On day 36, animals were euthanised. During the treatment period, acute or delayed mortality was checked every day.

### Endpoint Measurements

Elevated plus maze: On day 29, the anxious state of mice was measured by evaluating their ability to explore open and enclosed arms of an elevated plus maze. The clear plexiglass apparatus consisted of two open arms (23.5×8 cm) and two enclosed arms (23.5×8×20 cm high), extending from a central platform and placed 50 cm above the floor. Each mouse was placed at the center of the plus maze facing a closed arm and its exploration behaviour was recorded by Ethovision® XT 9.0 (Noldus Information Technology) for 10 min. The results were expressed as locomotor activity, time spent in the open arms, and number of open arm entries. The gravity center of animal was considered by Ethovision® XT 9.0 software to calculate the position of the animal in the elevated plus maze.

Open field: On day 30, mice were placed individually in a squared open-field (50 cm×50 cm×50 cm high) made from white plexiglass with a floor equipped with infrared light emitting diodes. Mice were habituated to the open-field for 10 minutes and their locomotor activity captured through an IR-sensitive camera and analysed using the Ethovision® XT 9.0 software (Noldus Information Technology). Behaviour was analysed as locomotor activity (distance traveled, cm), locomotor activity in the 25×25 cm central area defined by the software, and number of stereotypies (sum of the number of rearing and grooming episodes) presented by the mice.

Novel object recognition: On day 31, two identical objects (50 ml plastic vials with caps) were placed at defined positions (position #1 and position #2, at two opposite edges of the central area) of the open-field plexiglass arena. Each mouse was placed in the open-field and the exploratory activity recorded during a 10-min duration session. The activity was analysed using the nose tracking protocol, in terms of number of contacts with objects and duration of contacts. The results are expressed as percentage of object interactions and percentage of the total interaction duration with the object in position #2.

On day 32, the object in position #2 was replaced by a novel one differing in color shape and texture from the familiar object. Each mouse was placed again in the open-field and the exploratory activity recorded during a 10-min duration session. The activity was analysed similarly. The preferential exploration index was calculated as the ratio of the number (or duration) of contacts with the object in position #2 over the total number and duration of contacts with the two objects. Animals showing less than 10 contacts with objects during the sessions were discarded from the study.

Forced swim test: Behavioural despair, a measure of susceptibility to depression, was assessed using the forced swim test. Each mouse was placed individually in a glass cylinder (diameter 12 cm, height 24 cm) filled with water at a height of 12 cm. Water temperature was maintained at 22-23° C. The animal was forced to swim for 6-min. The session was recorded by a CCD camera connected to a computer and movements were analysed using Ethovision® XT 9.0 software (Noldus Information Technology). Two levels of pixel changes were analysed to discriminate between immobility, struggling, and swimming. Analyses were performed min per min the last five minutes of the procedure.

Forced swim test: Behavioural despair, a measure of susceptibility to depression, was assessed using the forced swim test. Each mouse was placed individually in a glass cylinder (diameter 12 cm, height 24 cm) filled with water at a height of 12 cm. Water temperature was maintained at 22-23° C. The animal was forced to swim for 6-min. The session was recorded by a CCD camera connected to a computer and movements were analysed using Ethovision® XT 9.0 software (Noldus Information Technology). Two levels of pixel changes were analysed to discriminate between immobility, struggling, and swimming. Analyses were performed min per min the last five minutes of the procedure.

Statistical analysis: Values were expressed as mean±Standard Error of Mean (SEM). Statistical analyses were performed using Prism 5.0a (GraphPad Software, Inc.) on the different conditions depending on results from Shapiro-Wilk normality test:
With a one-way ANOVA (F value), followed by the Dunnett's post-hoc multiple comparison test to compare individual groups to each other, if data followed a Gaussian distribution.
With a Kruskal-Wallis non-parametric ANOVA (H value), followed by a Dunn's multiple comparison test if data did not follow a Gaussian distribution.

p<0.05 was considered to be statistically significant.

### Example 3: Study—Characterisation of the Effect of LP12151 Versus LP12407 and LP12418 on Repeated Stress in Mice

### Study Protocol

Sixty Swiss mice (30 to 35 g) were used in this study. Five animal groups were constituted in the following manner, according to Table 1 below.

TABLE 1
Treatment groups

| | | n |
|---|---|---|
| 1. | No stress male mice/Vehicle | 12 |
| 2. | Chronic stressed male mice/Vehicle | 11 |
| 3. | Chronic stressed male mice/Lactobacillus plantarum LP12151, 10⁹ CFU/day | 12 |
| 4. | Chronic stressed male mice/Lactobacillus plantarum LP12407, 10⁹ CFU/day | 12 |
| 5. | Chronic stress male mice/Lactobacillus plantarum LP12418, 10⁹ CFU/day | 11 |

| | |
|---|---|
| Total mice | 58 |

Animals were randomly assigned to an experimental group, weighed and treated with the appropriate strain/vehicle in accordance with the methods set out in Example 2 above. From day 01 to day 33, animals were treated in accordance with the treatment schedule described in Example 2 above.

### Results and Comments

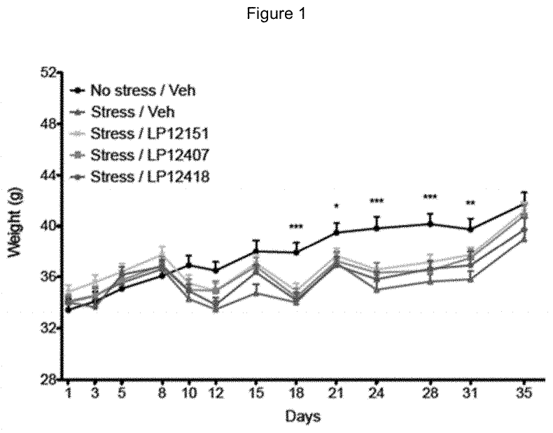

Animals weight: FIG. 1 shows the effects of chronic stress on body weight in mice. Chronic stress induced a non-significant difference of body weight from day 18 to day 32 of the study as compared to the no stress/vehicle treated group. The LP12151 strain reduced the loss of body weight as compared to chronically stressed mice in a non-significant manner.

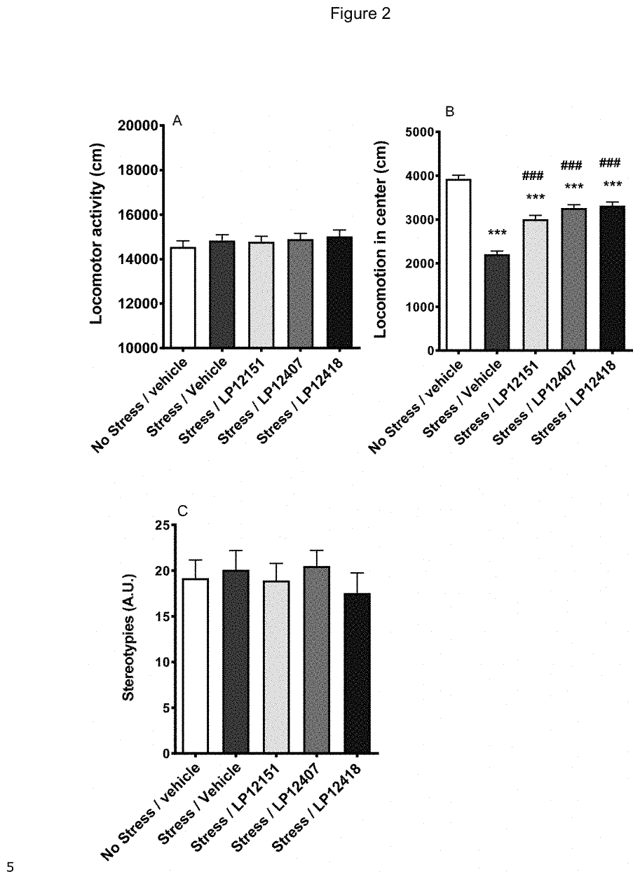

Anxiety in the open field procedure: FIG. 2 shows the effects of the different treatments on chronic stress-induced anxiety in mice. As shown in FIG. 2A, neither stress alone, nor any of the treatments, had an observable influence on locomotor activity. However, chronic stress did induce a very significant decrease of locomotion in the center of the arena; an indication that chronic stress induced an anxiety like behaviour (FIG. 2B). Furthermore, treatment with LP12151 partially but very significantly alleviated this deficit. Neither chronic stress alone nor any of the treatments showed any influence on the rearing/grooming behaviour, related to stereotypic activity (FIG. 2C).

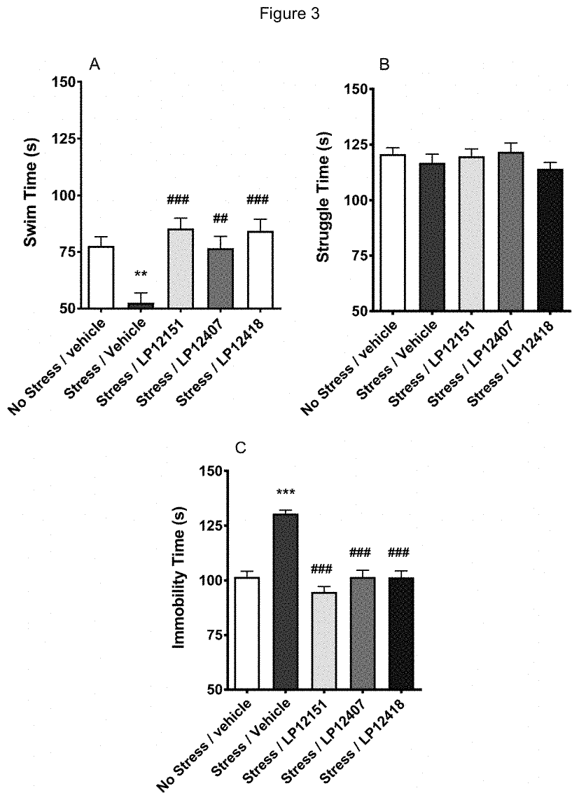

Forced swim test: FIG. 3 shows the effects of chronic stress and the various treatments on chronic stress-induced behavioural despair in mice. The forced swim test was used to measure three different parameters; immobility time (parameter A), struggle time (parameter B) and swim time (parameter C). Chronic stress induced an increase of immobility in the forced swim test (FIG. 3A). Chronic stress also reduced swim time compared to the no stress/vehicle group (FIG. 3C). LP12151 treatment fully alleviated these deficits. In terms of struggle time, neither chronic stress nor any of the various treatments appeared to influence this parameter compared to the no stress/vehicle group (FIG. 3B).

The following conclusions can be drawn from the results described above:

Chronic stress-induced behavioural deficits:
Induced a body weight decrease as compared to non-stressed mice.
Induced an anxiety-like state in mice in open field test.
Showed a significant depressive-like state observed by behavioural despair in the forced swim test.

Treatment with L. plantarum LP12151:
Reduced the loss of body weight as compared to chronically stressed mice in a non-significant manner.
Very significantly but partially reduced the anxiety-like state in mice in the open field test.
Showed a very significant antidepressant-like effect observed as behavioural despair in the forced swim test, compared to chronically stressed mice.

1. (canceled)

2. A method of preventing and/or treating a mental illness, a symptom affecting mental health and/or a condition associated with chronic stress in a mammal in need thereof, wherein the method comprises administering to the mammal Lactobacillus plantarum is strain LP12151 deposited with DSMZ on Jan. 9, 2018 under accession number DSM 32721.

3. The method according to claim 2, wherein:
the method comprises preventing and/or treating a mental illness; and
the mental illness is a mood disorder, an anxiety disorder and/or depression.

4. The method according to claim 2, wherein:
the method comprises preventing and/or treating a symptom affecting mental health; and
the symptom affecting mental health is anxiety, mood swings and/or depression

5. The method according to claim 2, wherein:
the method comprises preventing and/or treating a condition associated with chronic stress; and
the condition associated with chronic stress is a gastrointestinal disorder.

6. (canceled)

7. A composition comprising:
Lactobacillus plantarum, strain LP12151 deposited with DSMZ on Jan. 9, 2018 under accession number DSM 32721; and
a second ingredient selected from a nutritionally acceptable carrier, a nutritionally acceptable diluent, a nutritionally acceptable excipient, a nutritionally acceptable adjuvant, a nutritionally active ingredient, a dietary ingredient, a pharmaceutically acceptable carrier, a pharmaceutically acceptable diluent, a pharmaceutically acceptable excipient, a pharmaceutically acceptable adjuvant, and a pharmaceutically active ingredient.

8. A method of preventing and/or treating a mental illness, a symptom affecting mental health and/or a condition associated with chronic stress in a mammal in need thereof, wherein the method comprises administering to the mammal a composition of claim 7.

9. The composition according to claim 7, wherein the second ingredient comprises another bacterial strain.

10. The composition according to claim 7, wherein the second ingredient comprises a prebiotic.

11. The composition according to claim 10, wherein the prebiotic is polydextrose.

12. The composition according to claim 7, wherein the composition is in the form of a food product, a beverage, a dietary supplement or a pharmaceutically acceptable formulation or composition.

13. The composition according to claim 7, wherein the composition is a spray dried or freeze-dried composition.

14. The composition according to claim 13, wherein the composition comprises a cryoprotectant.

15. The composition according to claim 7, wherein:
the composition is in unit dosage form, and
the Lactobacillus plantarum strain LP12151 is present in the composition in an amount between 10⁶ and 10¹² CFU per unit dose.

16. The method according to claim 8, wherein the mental illness is a mood disorder, an anxiety disorder and/or depression.

17. The method according to claim 8, wherein the symptom affecting mental health is anxiety, mood swings and/or depression.

18. The method according to claim 8, wherein the condition associated with chronic stress is a gastrointestinal disorder.

19. A composition, wherein:
the composition comprises Lactobacillus plantarum strain LP12151;
the composition is spray dried or freeze-dried, and
the composition comprises a cryoprotectant.

20. The composition of claim 19, wherein the cryoprotectant comprises glucose, sucrose, lactose, trehalose or sodium ascorbate.

21. The method according to claim 5, wherein the gastrointestinal disorder comprises irritable bowel syndrome.

22. The composition according to claim 15, wherein:
the composition is in unit dosage form, and
the Lactobacillus plantarum strain LP12151 is present in the composition in an amount between 10⁸ and 10¹² CFU per unit dose.
